(19) United States
(12) Patent Application Publication
(10) Pub. No.: US 2026/0273283 A1
(43) Pub. Date: Sep. 17, 2026

(54) AUTOMATED PERCEPTION THRESHOLD TEST AND THERAPY ADAPTATION FOR SPINAL CORD STIMULATION

(71) Applicant: BIOTRONIK SE & Co. KG, Berlin (DE)

(72) Inventors: Luis Genehr Alves, West Linn, OR (US); David McIntosh, Wilsonville, OR (US); Andrew B. Kibler, Lake Oswego, OR (US); Rainer Joerg Grosskopf, Portland, OR (US)

(21) Appl. No.: 19/166,676

(22) PCT Filed: Jan. 30, 2024
(86) PCT No.: PCT/EP2024/052190

§ 371 Date: Sep. 18, 2025

Related U.S. Application Data
(60) Provisional application No. 63/453,804, filed on Mar. 22, 2023.

(30) Foreign Application Priority Data
Apr. 19, 2023 (EP) 23168759.1

(51) Int. Cl. A61N 1/36; A61N 1/372

(52) U.S. Cl. CPC A61N 1/36071; A61N 1/36062; A61N 1/36132; A61N 1/36139; A61N 1/37247

(57) ABSTRACT

A neurostimulation device with one or more electrodes for implanting into a patient is configured to determine a perception threshold, PT, of the patient at least in part based on patient feedback. The device is further configured to adjust at least one stimulation parameter at least in part based on the PT and a predetermined adjustment factor or adjust at least one therapeutic window of the neurostimulation device at least in part based on the PT.

The present invention relates to a neurostimulation device and a system configured to determine a perception threshold, a respective system, respective methods, and computer programs.

Neurostimulation devices may be used for various kinds of neurostimulation, for example for therapeutic purposes like chronic pain treatment. Typically, neurostimulation pulses are provided to the neuronal system of a patient via one or more electrodes of the neurostimulation device. For this, it may be suitable to place the electrodes of the neurostimulation device in proximity to the spinal cord of the patient for so-called spinal cord stimulation (SCS).

The aim of a neuromodulation device for pain is to selectively target nerve fibers responsible for transmitting the sensation of pain. However, over-stimulation may also fire motor/sensory nerve fibers which the patient can feel. The minimum amplitude at which a signal produces a response that the patient can feel is called Perception Threshold (PT). Generally, the (PT) of a patient is seen as a suitable reference point for the stimulation amplitude of the neurostimulation device.

However, the PT of the patient may vary over time due to, e.g., positional shifts of the electrodes, physiological changes, potentiation, and/or habituation. Setting the neurostimulation parameters, based at least in part on an initially determined PT, does generally not account for potential changes of the PT. The neurostimulation provided to the patient may thus become ineffective, under- or overstimulating and/or potentially even harmful. Any such scenario may result in patient discomfort, return of pain, excessive energy use, and discontent.

Moreover, adjustment procedures of parameters like the PT setting of a neurostimulation device may require time-consuming appointments with a health professional to re-set the required parameters. These sporadic appointments may further mean that the device cannot provide the optimal therapy for possibly several days, weeks, or months in the time between appointments.

Altogether, there is therefore still a need to further improve neurostimulation devices.

The above need is at least partly met by the aspects described herein.

In a first aspect, a neurostimulation device with one or more electrodes for implanting into a patient may be provided wherein the neurostimulation device may comprise means for determining a perception threshold, PT, of the patient at least in part based on patient feedback. Moreover, the neurostimulation device may comprise: Means for adjusting at least one stimulation parameter at least in part based on the PT and an adjustment factor, and/or for adjusting at least one therapeutic window at least in part based on the PT.

The patient feedback may be provided via conscious feedback. In an exemplary process, a neurostimulation amplitude may be varied from a first value to a second value, possibly in multiple steps or in a (quasi-) continuous way. The patient may be instructed to provide feedback when the neurostimulation varying in amplitude exceeds the PT, e.g., to indicate that the patient perceives the stimulation, and/or to provide feedback when the varying neurostimulation amplitude falls below the PT, e.g. to indicate that the patient does not perceive the stimulation anymore. Patient feedback in either direction may be used to determine the PT accordingly. The neurostimulation device may process such patient feedback in combination with data, based at least in part on the provided neurostimulation, to determine the PT. The patient may give feedback via a remote device, e.g., a remote specifically provided for the neurostimulation device, a mobile phone via an according application, a computer, or any other device capable of communicating with the neurostimulation device. This may be particularly advantageous to include the patient to increase patient satisfaction and to reduce patient discomfort as such PT determination may be performed at any time and independently from a health professional, effectively reducing periods during which neurostimulation may be in an unsuitable setting.

In a further example, once the PT is determined, the neurostimulation device may adjust at least one stimulation parameter, for example the stimulation amplitude at least in part based on the PT to a value of, e.g., the PT multiplied by a predetermined adjustment factor. Hence, an ideal setting value dependent on the PT may be provided for the stimulation parameter based on the current PT (provided by patient feedback) and the predetermined adjustment factor (which may reflect predetermined treatment parameters). For example, the stimulation amplitude may be set to more than the PT or less than the PT.

Additionally or alternatively, the neurostimulation device may adjust at least one therapeutic window at least in part based on the PT. The at least one therapeutic window may comprise sub- and/or above-PT values, e.g., defining a sub-PT and/or an above-PT window. Generally, the therapeutic window may define a range within which a stimulation parameter may be selected by the neurostimulation device during operation. For example, the neurostimulation device may be adapted to adjust the stimulation parameter based on predetermined patient preferences, patient input during operation, and/or any other parameter or external signaling (e.g. from a remote device as outlined herein). This may provide stimulation therapy tailored to the patient's needs while at the same time ensuring that the at least one stimulation parameter remains within safe limits, preventing under- or overstimulation of the patient, for example.

In some examples, in which the therapeutic window is determined, the feedback may not necessarily be based on manual patient feedback. For example, the patient feedback may at least in part be based on evoked compound action potentials, ECAPs. These may be measured by one or more electrodes implanted into the patient. Potentially, these electrodes may be electrodes of the neurostimulation device, or even the same electrodes used to provide neurostimulation. Such feedback may be advantageous as it may be provided without participation of the patient in a convenient, patient-friendly, and possibly fully automated way.

According to an embodiment, the means for determining the PT of the patient are adapted for crossing the PT a plurality of times for determining a plurality of PT values and for determining the PT at least in part based on the plurality of PT values.

Performing consecutive threshold processes that are designed to cross the PT may be particularly helpful as the patient may localize the area of perception in a first threshold process and is prepared to provide even more accurate feedback from the second threshold process onward. This may improve reliability of the PT determination, makes the device more user/patient-friendly and allows for further functionalities reliant on multiple PT values determined within one threshold process sequence. Apart from that, also a validation may be carried out, e.g., a check whether first and second values are consistent with each other, etc.

In an example, the neurostimulation device may be configured to cross the PT multiple times in a process in which the neurostimulation amplitude may be varied from a first value to a second value, as described herein, e.g., in a threshold process. The first value might be higher or lower than the second value. Typically, one value may be higher than the (previous and/or currently expected) PT and one value may be lower than the PT to ensure that the PT is crossed. In a simple example, the PT may be crossed in multiple identical consecutive threshold processes/scans. The consecutive threshold processes, however, may also differ in at least one aspect. For example, the scanning direction may change between threshold processes, i.e., the first threshold process crosses the PT from a first to a second value, the second threshold process crosses the PT from the second to the first value, and so an. Further, any other scanning pattern with potentially even different first and second values associated with each threshold process may be realized. In particular, the first and/or the second value of a threshold process may be adapted in a subsequent threshold process, e.g. if no patient feedback is received for the threshold process, potentially indicating that the PT was not crossed, i.e., the PT was not enclosed in the range between the first and the second value.

Generally, feedback used to determine the PT may comprise (conscious) patient feedback, as described herein, or feedback based on measuring ECAPs via at least one electrode or any other suitable detector/sensor. Hence, according to an embodiment, the means for determining a PT of the patient is at least in part based on patient feedback in form of an ECAP, measurement.

Moreover, according to an embodiment, the means for adjusting at least one stimulation parameter of the neurostimulation device is configured to multiply the PT with the adjustment factor, wherein the adjustment factor is a percentage value. For instance, the adjustment factor can be in the range of 0.1 to 0.95, or 1.05 to 1.5. Alternatively or in addition, the means for adjusting are adapted to adjust the at least one therapeutic window to a range within 0.1 to 0.95 PT or 1.05 to 1.5 PT.

A further aspect, that may be implemented independently from but also in combination with the aspects described herein may be a neurostimulation device which may not necessarily determine the PT itself. For example, it may be programmed to implement a varying stimulation amplitude (e.g. corresponding to a threshold process as described herein) which may be initialized a plurality of times, e.g., by a remote device as described herein. The remote device may be configured to trigger the variation in the stimulation amplitude and determine the PT based on (patient) feedback. The neurostimulator may then be instructed accordingly, e.g. to adjust at least one stimulation parameter at least in part based on the PT and a predetermined offset from the PT and/or to adjust at least one therapeutic window at least in part based on the PT. Additionally or alternatively, the remote device may comprise an interface for providing a suggestion for at least one stimulation parameter and/or for at least one therapeutic window of the neurostimulation device at least in part based on the PT. Upon a subsequent user input, the neurostimulation device may then be instructed accordingly. Additionally or alternatively, the remote device may be adapted for letting the neurostimulation device cross the PT a plurality of times for determining a plurality of PT values, e.g. by correspondingly triggering it. The remote device may also comprise means for determining the PT at least in part based on the plurality of PT values.

According to an embodiment of the present invention, the neurostimulation device is configured to determine the PT in a threshold process. The threshold process comprises the steps of:
a) applying a first stimulation to the patient according to at least one first test in 5 stimulation parameter;
b) receiving feedback for the first test stimulation,
c) increase the stimulation amplitude for the first test stimulation if the feedback comprises no sensation in response to the first test stimulation,
d) repeating steps a)-c) until the feedback comprises a sensation in response to the first test stimulation.

The proposed exemplary threshold process ensures an iterative step by step process to approach the PT of a patient. The patient feedback can for instance be provided by a manual feedback process, e.g. by entering the patient feedback information via an interface of a remote device. The remote device and the neurostimulation device have a communication connection, and the stimulation parameter of the neurostimulation device can be either manually or automatically controlled by the remote device. Alternatively or in addition, the patient feedback can be provided by ECAP measurements from sensors, which automatizes the patient feedback loop.

Generally, any functionality described herein with reference to the neurostimulation device may not necessarily be provided by the neurostimulation device itself, but it may also be provided by a remote device as described herein, e.g. a patient device (such as a remote control, a smartphone, etc.), a server and/or other centralized computing device that may be in communication with the patient device and/or the neurostimulation device. In an example, for a system comprising the neurostimulation device to be able to perform any function described herein, any such function or any part of such function may be implemented by the server, the remote device and/or the neurostimulation device, as needed. This may in one example provide a neurostimulation device only configured to execute functions as fully instructed by an external device. The patient device and/or the server may comprise means for receiving signaling from and/or providing signaling to the neurostimulation device directly or indirectly. The neurostimulation device may comprise means for receiving signaling from and/or provide signaling to the patient device directly or indirectly. In some examples, the server and/or the patient device may be configured to monitor, determine and/or change the neurostimulation parameters, the offset from the PT, the therapeutic window and/or to monitor and/or determine the PT.

In some examples, the means for adjusting may be adapted to adjust the at least one stimulation parameter to a value in the range of 0.1 to 0.95 PT or 1.05 to 1.5 PT and/or to adjust the at least one therapeutic window to a range within 0.1 to 0.95 PT or 1.05 to 1.5 PT.

For example, setting the at least one stimulation parameter, e.g., the stimulation amplitude, to a value in the above-identified range provides the possibility to deliver sub-PT stimulation to the patient when in the range from 0.1 PT to 0.95 PT and provides the possibility to deliver above-PT stimulation to the patient when in the range from 1.05 PT to 1.5 PT.

In an example, adjusting the at least one therapeutic window to the above-identified range may limit the range in which the respective at least one stimulation parameter may be set by the patient, a health professional or the neurostimulation device itself in an automatic way, which may improve safety by ensuring that neurostimulation remains within safe limits preventing harmful (over-)stimulation to the patient.

In a further example, the neurostimulation device may comprise an interface for receiving patient input to adjust the at least one stimulation parameter within the therapeutic window. Thus, the patient may quickly adjust the stimulation according to their needs to keep neurostimulation at a suitable setting at any time which may improve patient satisfaction and reduce patient discomfort.

For example, this adjustment of the at least one stimulation parameter may be done by the patient at any time, independent from the determination of the PT. The patient may, for example, be able to adjust the value within the therapeutic window. The neurostimulation device may in an example provide predetermined suggestions, e.g., a “high”, a “medium”, and a “low” option, to the patient, for example via a remote device. Alternatively or in addition, the patient may be free to set any stimulation parameter via the interface, e.g. overriding the suggestion etc.

In a further example, the neurostimulation device may further be configured to determine the PT in a threshold process comprising applying a first stimulation to the patient according to at least one first test stimulation parameter, applying a second stimulation to the patient according to at least one second test stimulation parameter, and receiving feedback for at least one of the first and second stimulation.

For example, any difference between the at least one test stimulation parameter differing between a first and a second stimulation of a threshold process may be adjusted as described herein. It is to be understood that a threshold process may comprise further stimulations according to at least one further test stimulation parameter. The total number of stimulations is not limited and may be larger than two. The plurality of stimulations applied to the patient, from a first stimulation according to at least one first test stimulation parameter until the last stimulation according to at least one last test stimulation parameter, may form a ramp from the first stimulation to the last stimulation of the threshold process. Generally, when differences between two consecutive stimulations are small, high sensitivity can be achieved. When differences between two consecutive stimulations are large, high scanning speeds can be achieved, suitable to scan a wider range of at least one stimulation parameter. Any combination of step width and scanning speed may be adjusted to the requirements of the respective threshold process. These parameters may be adjusted to data at least partly based on previous threshold processes. For example, when the patient previously recorded only moderate changes in the PT between consecutive threshold processes, it may be suitable to perform the threshold process with a small step width (and a low scanning speed) across only a narrow range as it may be expected that the new PT value lies therein and can be determined with high accuracy. Further, the at least one parameter may be increased or decreased in some examples at least until (patient) feedback is provided to ensure that the PT value is crossed. In some examples, when large variations in the PT were previously determined, it may be advantageous to scan a wider range with a large step width (at a high scanning speed). This may allow for an ideal adjustment of the PT determination process according to the patient's needs.

In a further example, the neurostimulation device may be configured to carry out at least two threshold processes for determining the PT. The neurostimulation may, optionally, further be configured to determine and/or validate the PT at least in part based on the at least two threshold processes.

In an example, such threshold process may comprise a first, a second and any further plurality of consecutive stimulations to the patient, as outlined herein. In an example, the same threshold process may be carried out more than once, e.g. to obtain a more accurate result, e.g. based on averaging. Similarly, a cross-check may be made to exclude that, e.g. the value determined in a first threshold process may be an outlier. However, despite being in principle the same threshold process, each threshold process may be configured to be halted, as soon as feedback (e.g. indicating threshold crossing) is received, such that each threshold process may in principle be ended at a different point in time.

However, it is also possible that, for example, the step-width, the scanning speed, the scanning direction may vary between consecutive threshold processes. In particular, the step width and/or scanning speed may be reduced in the range in which the PT may be expected, e.g., at least in part based on the PT determined in previous threshold process sequences and/or previous threshold processes of the same threshold process sequence. This may improve sensitivity and result in a more accurate determination of the PT improving the over-all adjustment of stimulation parameters.

In an example, at least two threshold processes may be used which comprise different scanning directions. For example, a first threshold process may start from a relatively low value of a stimulation parameter (e.g., stimulation amplitude) which may increase to a relatively higher value. A second threshold process may, in turn start from a relatively high value of a stimulation parameter (e.g., stimulation amplitude) which may decrease to a relatively lower value. Hence, the threshold is expected to be crossed from different directions, which may lead to a more accurate overall result.

In an exemplary determination of the PT, at least in part based on the at least two threshold processes, the PT may be determined by an average of the PT values determined in the at least two threshold processes. Any other statistical analysis may be implemented to determine the PT.

In a further example, the determination of the PT, at least in part based on the at least two threshold processes, may involve a validation of the acquired PT. This validation may be based on comparisons of the determined PT values of consecutive threshold processes or a plurality of threshold process sequences, and/or the according statistically determined PT values. In some examples, the determined PT may be rejected based on the validation. For example, variations in the PT values above a given limit may result in the rejection. Any validation described herein may also occur prior to, after and/or simultaneously with any statistical analysis as described herein.

Overall, this may result in improved reliability of the PT determination and the prevention of faulty PT values, e.g., due to errors by the patient during the PT determination, which might result in an unsuitable and potentially even harmful neurostimulation.

It is noted that parameters such as step size, scan speed may also vary within each threshold process.

In a further example, the neurostimulation device may be configured such that the at least one stimulation parameter described herein comprises at least one of an amplitude, a waveform type, a pulse width, a frequency, and/or a duty-cycle.

Adjusting at least one of these parameters may result in a higher degree of freedom in optimizing the therapy in adjustment to the patient's needs, increasing battery lifetime, reducing the energy consumption, and any combination thereof. Notably, not only the amplitude, but also a waveform type, a frequency, and/or a duty-cycle may be adapted for example at least in part based on the PT. For example, at least one value of the amplitude, the waveform type, the frequency, and/or the duty-cycle may be comprised in the suggestion provided to the patient, the limits of the at least one therapeutic window, the stimulation provided to the patient, etc.

In a further example, the neurostimulation device may further be configured to provide the PT to a remote device. This may enable a monitoring of the PT value(s), e.g., by a server, and/or by medical staff, increasing safety.

In an example, other data related to the operation of the neurostimulation device may be provided to the remote device as well. The remote device may for example be a server. It may monitor operation of the neurostimulation device.

In a further aspect, a system is proposed, comprising the neurostimulation device according to any previously embodiment described, and a remote device for communication with a neurostimulation device may be provided which comprises an interface for receiving patient input, means for monitoring the PT, and means for providing at least one stimulation parameter and/or means for controlling at least one stimulation parameter at least in part based on the monitoring of the PT.

In some examples, the interface may be part of the remote device, e.g. any remote device associated with the patient, e.g. a remote (control) device of the neurostimulation device, a smart phone, and/or in general any data-processing device for providing a suggestion for at least one stimulation parameter may be adapted to provide the suggestion. These devices may be configured to communicate the suggestion for at least one stimulation parameter to the patient via the interface, e.g. via one or more audio signals, visual signals, haptic signals, and/or any combination thereof. For example, the device involved in communicating the suggestion for at least one stimulation parameter may be the same device that is also used for patient feedback. The device may further comprise a user interface to allow the user to input a stimulation parameter at least in part based on the suggested parameter/window and/or to input a therapeutic window at least in part based on the suggested parameter/window. This may improve safety and reliability in setting the stimulation parameters combined with giving the patient the freedom to choose at the same time. Thus, this may also improve patient satisfaction and reduce patient discomfort.

Monitoring the PT, and thus any parameter at least in part based on the PT, may be an advantageous and convenient safety measure to ensure that the neurostimulation therapy provided to the patient remains within an effectual range, even under potential adaptation to a varying condition of the patient. This may be done remotely without time-consuming and work-intensive appointments with a health professional.

According to an embodiment of the proposed system, the neurostimulation device is configured to provide the PT to a remote device. The means for determining a PT of the patient is at least in part based on patient feedback in form of an ECAP measurement.

According to an embodiment of the inventive system, the interface of the remote device is configured to provide means for adjusting the at least one stimulation parameter within the therapeutic window.

In an example, the remote device may be a patient device as described herein. It may be configured to provide an interface for patient feedback and/or for providing the patient with information based at least in part on the status of the neurostimulation device and/or past, planned and/or currently running threshold processes, the PT, any stimulation parameter, the at least one therapeutic window and any other parameter and/or setting described herein. The signaling may in an example occur via wiring and/or in a wireless way. The remote device may provide a user-friendly way to provide a device for entering user feedback and/or receiving any information related to the neurostimulation device.

In an exemplary embodiment, the remote device may additionally or alternatively comprise a server. The server may be configured to provide signaling to a health professional and/or the neurostimulation device. For example, the server may be configured to set one or more parameters associated with the neurostimulation therapy applied by the neurostimulation device and/or threshold process(es) to determine the PT. The neurostimulation device may be configured to perform functions as described herein according to the instructions received from the server. At the same time, the health professional may be informed on the processes performed by the neurostimulation device as well as their outcomes, e.g., the determined PT. In an example, the neurostimulation device may also provide the patient feedback independently from any external device.

In a further exemplary embodiment, the neurostimulation device may be configured to initiate and/or instruct its means to determine the threshold itself. It may operate in such a way that its interaction with the server may comprise providing the server with information based at least in part on the PT, e.g., for the server to archive these data for monitoring by a health professional or any automated data processing and/or monitoring.

According to an embodiment of the present system, the interface of the remote device is configured to receive the feedback for the first test stimulation from a user. The interface is configured to provide means for increasing the stimulation amplitude for the first test stimulation if the feedback comprises no sensation in response to the first test stimulation.

Generally, any exemplary system may provide an advantageous distribution of the required functionalities to the different devices and/or means of the system. As described herein, any functionality and/or feature of the system may be implemented in either the neurostimulation device and/or the remote device (e.g. a patient device and/or a server).

In a further aspect, a method for a neurostimulator with one or more electrodes for implanting into the patient may be provided. The method may comprise determining the PT of the patient at least in part based on patient feedback. It may further comprise adjusting at least one stimulation parameter at least in part based on the PT and a predetermined adjustment factor, and/or adjusting at least one therapeutic window at least in part based on the PT.

The method may be implemented by the neurostimulation device, the remote device and/or the system comprising both. The method may for example provide a convenient way for the patient to adjust the PT and thus the stimulation parameters according to their needs whenever needed such that periods in which unsuitable neurostimulation is provided to the patient are reduced.

According to an embodiment, the inventive method further comprises determining the PT of the patient, providing a suggestion for at least one stimulation parameter at least in part based on the PT and/or providing a suggestion for at least one therapeutic window at least in part based on the PT.

The method may, e.g., be implemented according to any example described herein in reference to the neurostimulation device, remote device and/or the system. The method may, for example, provide an interactive option for the patient to adjust the stimulation according to their need assisted by the suggestion provided. This may improve patient satisfaction and prevent faulty settings due to patient input.

According to an aspect, the proposed method further comprises the steps of
a) applying a first stimulation to the patient according to at least one first test stimulation parameter (210);
b) receiving feedback for the first test stimulation,
c) increase the stimulation amplitude for the first test stimulation if the feedback comprises no sensation in response to the first test stimulation,
d) repeating steps a)-c) until the feedback comprises a sensation in response to the first test stimulation.

In a further aspect, a method for a neurostimulation device with one or more electrodes for implanting into a patient may be provided. The method may comprise determining the PT of the patient and crossing the PT a plurality of times for determining a plurality of threshold values and determining the PT at least in part based on the plurality of threshold values.

The method may, e.g., be implemented according to any example described herein in reference to the neurostimulation device, remote device and/or the system.

Further, any combination of the methods according to the aspects described herein may be realized as well. The methods, the remote device, the system and/or the neurostimulation device according to any aspects described herein may further comprise steps and/or be configured, respectively, to provide feedback to the patient comprising instructions and/or information based at least in part on the PT determination. This information may comprise information on whether the stimulation amplitude is increasing or decreasing, if the threshold process is in progress or if normal operation is in progress, etc.

Further, any functionality described herein may be implemented as means of a corresponding device, one or more steps of a corresponding method and/or one or more instructions of a corresponding computer program, even if it expressly only described with reference to a device, and/or method.

The devices and methods are described in the description herein and illustrated in the accompanying drawings by various blocks, components, circuits, processes, algorithms, etc. (collectively referred to as “means”). These means may be implemented using electronic hardware, computer software, or any combination thereof. Whether such means are implemented as hardware or software depends upon the particular application and design constraints imposed on the overall system.

By way of example, a means, or any portion of a means, or any combination of means may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more example embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

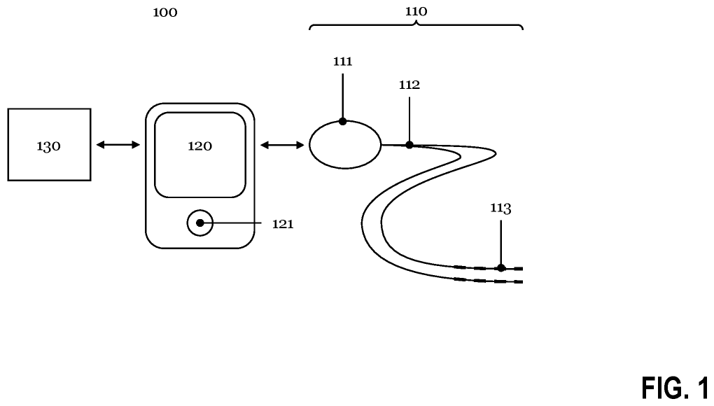

FIG. 1 shows an exemplary system comprising a neurostimulation device with electrode leads, a remote device and a server.

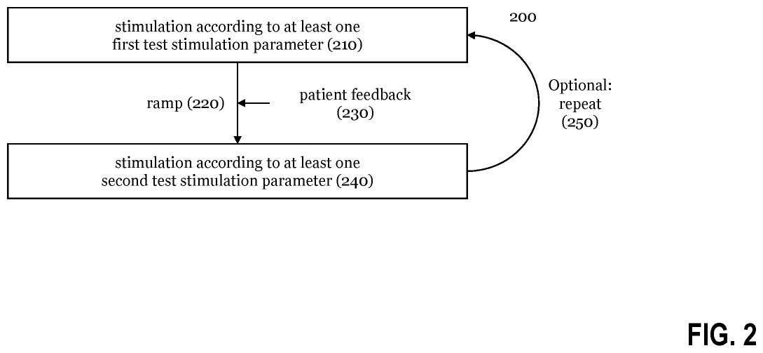

FIG. 2 is a flow chart of an exemplary threshold process.

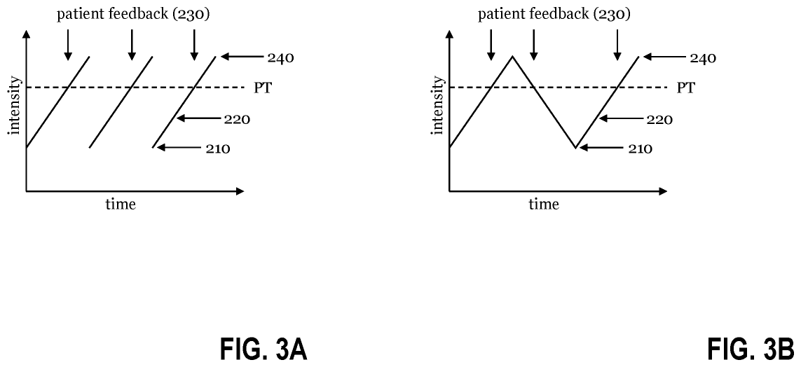

FIG. 3A shows the stimulation intensity over time for a first exemplary threshold process sequence.

FIG. 3B shows the stimulation intensity over time for a second exemplary threshold process sequence.

FIG. 1 shows an exemplary system 100 comprising a neurostimulation device 110, a remote device 120, and a server 130.

The neurostimulation device 110 of FIG. 1 comprises two leads 112 with electrodes 113 configured to be implanted into the patient, typically they are placed in the epidural space between the spinal cord and the vertebrae. The neurostimulator device 110 itself is either an implantable pulse generator which is configured to be implanted subcutaneously, or an external stimulator, for instance for trial stimulation purposes. The neurostimulator further comprises a pulse generator 111, which typically comprises a battery and means to provide electric stimulation to the patient via the electrodes 113. In case the neurostimulation device is an implanted device, the pulse generator 111 may either be implanted into the patient as well, usually in the upper buttocks of lower back. Alternatively, in case the neurostimulation device 110 is an external device, it is fixated on the skin, wherein only the leads 112 are implanted in the patient. Generally, other examples of neurostimulation devices 110 may comprise more or fewer leads 112 and/or electrodes 113.

The arrows in FIG. 1 indicate possible communication paths. In this exemplary embodiment, the remote device 120 may be implemented as a patient device (e.g. remote control, smartphone, etc.) and configured to communicate with the server 130 and the neurostimulation device 110. In other systems 100, however, the neurostimulation device 110 might also directly communicate with the server 130.

In some examples, at least one threshold process may be performed by the neurostimulation device 110. The remote device 120 may be configured to allow the patient to provide patient feedback via, e.g., a button 121 of the remote device 120. Alternatively or in addition, the neurostimulation device 110 may receive that patient feedback, in case the patient feedback is measure by sensors in the form of ECAPS. Alternatively, the patient feedback is received manually via the interface of the remote device 120. The PT is determined based on the patient feedback. According to an exemplary aspect of the invention, the remote device 120 would then be used to adjust at least one stimulation parameter of the neurostimulation device 110 at least in part based on the PT and a predetermined offset from the PT. Further, it may adjust at least one therapeutic window of the neurostimulation device 110 at least in part based on the PT.

For example, the neurostimulation device 110 may alternatively or in addition to adjusting at least one stimulation parameter of the neurostimulation device 110 at least in part based on the PT and a predetermined offset from the PT and/or adjusting at least one therapeutic window of the neurostimulation device 110 at least in part based on the PT, provide suggestions for the above-mentioned parameters. This may, e.g., be done via a user interface, e.g. a screen, speaker, etc., of the remote device 120. In some examples, also the neurostimulation device 110 itself may comprise an interface suitable for that matter, e.g. a user interface.

In some examples, the neurostimulation device 110 may be configured to cross the PT a plurality of times in a plurality of threshold processes 200. In this way, a plurality of PT values may be determined and determining the PT may at least in part be based on the plurality of PT values. The neurostimulation device may, e.g., process the plurality of PT values and provide the patient with information on the validity and/or the average PT value via the remote device 120.

The communication between the individual parts of the system 100 may be either unidirectional or bidirectional. For example, the server 130 might, in an exemplary system 100 with unidirectional communication between the remote device 120 and the server 130, be configured to only receive signaling from the remote device 120, e.g., comprising information on the PT. In other examples, the server 130 may also send signals to the remote device 120, e.g. to trigger PT determination, to adjust a frequency of PT determination, to provide information to the patient, and/or for remote programming of the neurostimulation device 110 etc.

In some examples, the communication between the neurostimulation device 110 and the remote device may be, e.g., bidirectional: For example, the remote device 120 may send instructions to the neurostimulation device 110 or the remote device 120 to perform a threshold process 200 and, e.g., receive information on the PT.

The exemplary remote device 120 shown FIG. 1 may comprise a screen and a button 121 via which the patient may start and stop a threshold process 200 and/or a threshold process sequence, provide patient feedback, etc. The screen may provide the patient with information the remote device 120 receives either from the server 130 and/or the neurostimulation device 110, or the patient feedback can be directly communicated between patient and clinician/user. For instance, the clinician ramps up the stimulation amplitude, and the patient tells the clinician as soon as she/he perceives a sensation. This information might, e.g., be at least in part based on the PT, current and/or previous threshold processes, reminders to determine the PT, and/or other information on the health status of the patient, potentially determined via other medical devices (as for instance implantable or non-implantable sensors) or other devices (for instance a wearable device as a smart watch).

FIG. 2 shows a flow chart of an exemplary threshold process 200. The exemplary threshold process 200 begins with a stimulation according to at least one first test stimulation parameter 210. The at least one stimulation parameter may in an example be the stimulation intensity/amplitude. The stimulation may then be applied according to the at least one second stimulation parameter 240. In an example, the stimulation amplitude may be increased from a first lower amplitude to a second higher amplitude. Preferably the lower amplitude is below the (expected) PT and the second amplitude is above the (expected) PT. In the exemplary threshold process 200, patient feedback 230 may be given by the patient, e.g., via a remote device indicating that the patient perceives the stimulation, e.g. for the first and/or the second stimulation, and the PT is surpassed accordingly. To perform not only one threshold process 200 but a threshold process sequence of at least two threshold processes 200, the first threshold process 200 may be repeated 250. However, as outlined herein, one or more parameters of the second and/or further threshold processes 200 may also be altered.

The stimulation amplitude may be increased according to a ramp 220 on which the at least one first and the at least one second stimulation amplitudes are located. However, such ramp 220 is not mandatory.

FIG. 3A shows the stimulation intensity over time for a first exemplary threshold process sequence. This first exemplary threshold sequence repeats the identical threshold process 200 two times, and optionally a third time. Starting from a stimulation according to at least one first test stimulation parameter 210 with a low stimulation amplitude, a stimulation according to at least one second test stimulation parameter 240 is applied with a higher stimulation amplitude. The at least one first and second test stimulation parameters may be according to a ramp 220 which, in the example of FIG. 3A follows a linear course. As described herein, the ramp 220 may comprise any plurality of stimulations, i.e., more than two, which are applied according to at least one test stimulation parameter each.

The ramp 220 in other exemplary processes may also follow another course than the linear increase shown in FIG. 3A. It does not necessarily have to be a (quasi)-continuous stimulation. It may also be a sequence of individual stimulations (e.g., pulses). Additionally or alternatively, the ramp 220 may follow, e.g., a stepwise increase or any other non-linear course. In some examples, it may also comprise a negative slope. The point in time when patient feedback 230 is provided may be directly linked to the intensity of the neurostimulation applied at that time (or shortly before) and therefore used to determine the PT.

As outlined, also different parameters may be used for example in subsequent threshold processes 200, and the fact that the processes in FIG. 3A are identical is only exemplary.

FIG. 3B shows the stimulation intensity over time for a second exemplary threshold process sequence. In the exemplary threshold process sequence, the intensity is alternatingly increased and decreased in two consecutive threshold processes 200, and an additional optional third threshold process 200. Similarly, as outlined with reference to FIG. 3A, also other ramps 220 and/or differing ramps 220 may be used.

In detail, the threshold process 200 either starts from a stimulation according to at least one first test stimulation parameter 210 with a relatively low stimulation amplitude and performs a ramp 220 comprising the stimulation according to at least one second test stimulation parameter 240 in the first and the third threshold process 200 shown in FIG. 3B or the other way round in the second threshold process 200 shown in FIG. 3B.

The patient feedback 230 in the exemplary threshold process sequence may provide information about when the patient begins to perceive the neurostimulation in the first and third threshold processes 200 and about when they do not perceive it anymore in the second threshold process 200 when the intensity decreases. Generally, both information may be translated into the intensity corresponding to the PT. The following listing of claims will replace all prior versions, and listings, of claims in the application:

1. A neurostimulation device (110) with one or more electrodes (113) for implanting into a patient, comprising:
(a) means for determining a perception threshold, PT, of the patient at least in part based on patient feedback (230); and
(b) means for one or both of:
(i) adjusting at least one stimulation parameter of the neurostimulation device (110) at least in part based on the PT and a predetermined adjustment factor, or
(ii) adjusting at least one therapeutic window of the neurostimulation device (110) at least in part based on the PT.

2. A neurostimulation device (110) according to claim 1, wherein the means for determining are adapted for crossing the PT a plurality of times for determining a plurality of PT values and determining the PT at least in part based on the plurality of PT values.

3. The neurostimulation device (110) of claim 1, wherein the means for adjusting at least one stimulation parameter of the neurostimulation device is configured to multiply the PT with the adjustment factor, wherein the adjustment factor is a percentage value.

4. The neurostimulation device (110) of claim 3, wherein one or both of:
(i) the adjustment factor is a value in the range of 0.1 to 0.95 or 1.05 to 1.5, or
(ii) the means for adjusting are adapted to adjust the at least one therapeutic window to a range within 0.1 to 0.95 PT or 1.05 to 1.5 PT.

5. The neurostimulation device (110) of claim 1, wherein the means for determining a PT of the patient is at least in part based on patient feedback (230) in form of an Evoked Compound Action Potential, ECAP, measurement.

6. The neurostimulation device (110) of claim 1, further configured to determine the PT in a threshold process (200) comprising:
applying a first stimulation to the patient according to at least one first test stimulation parameter (210);
receiving feedback for the first test stimulation;
increasing the stimulation amplitude for the first test stimulation if the feedback comprises no sensation in response to the first test stimulation; and
d) repeating the steps of applying, receiving, and increasing until the feedback comprises a sensation in response to the first test stimulation.

7. The neurostimulation device (110) of claim 6, wherein the neurostimulation device (110) is configured to carry out at least two threshold processes (200) for determining the PT; and wherein the neurostimulation device (110) is further configured to one or both of:
(i) determine the PT at least in part based on the at least two threshold processes (200), or
(ii) validate the PT at least in part based on the at least two threshold processes (200).

8. The neurostimulation device (110) of claim 1, wherein the at least one stimulation parameter comprises at least one of an amplitude, a waveform type, a pulse width, a frequency, or a duty-cycle.

9. A system (100) comprising:
(a) a neurostimulation device (110) of claim 1, and
(b) a remote device (120) for communication with a neurostimulation device (110), wherein the remote device (120) comprises:
(i) an interface for receiving user input;
(ii) means for monitoring the PT; and
(iii) one or both of:
(A) means for providing at least one stimulation parameter, or
(B) means for controlling at least one stimulation parameter at least in part based on the monitoring of the PT.

10. The system (100) of claim 9, wherein the neurostimulation device (110) is configured to provide the PT to a remote device (120), wherein the means for determining a PT of the patient is at least in part based on patient feedback (230) in form of an ECAP measurement.

11. A system according to claim 9, wherein the interface is configured to provide means for adjusting the at least one stimulation parameter within the therapeutic window.

12. A system according to claim 9, wherein the interface is configured to receive the feedback for the first test stimulation from a user, and wherein the interface is configured to provide means for increasing the stimulation amplitude for the first test stimulation if the feedback comprises no sensation in response to the first test stimulation.

13. A method for controlling a neurostimulation device (110) with one or more electrodes (113) for implanting into a patient, the method comprising:
determining a perception threshold, PT, of the patient at least in part based on patient feedback (230); and
one or both of:
adjusting at least one stimulation parameter of the neurostimulation device (110) at least in part based on the PT and a predetermined adjustment factor, or
adjusting at least one therapeutic window of the neurostimulation device (110) at least in part based on the PT.

14. The method according to claim 13, further comprising crossing the PT a plurality of times for determining a plurality of threshold values and determining the PT at least in part based on the plurality of threshold values.

15. The method according to claim 13, further comprising determining the PT in a threshold process (200) comprising:
applying a first stimulation to the patient according to at least one first test stimulation parameter (210);
receiving feedback for the first test stimulation;
increasing the stimulation amplitude for the first test stimulation if the feedback comprises no sensation in response to the first test stimulation; and
d) repeating the steps of applying, receiving, and increasing until the feedback comprises a sensation in response to the first test stimulation.
